(19) United States
(12) Patent Application Publication
(10) Pub. No.: US 2022/0080069 A1
(43) Pub. Date: Mar. 17, 2022

(54) Card Sanitizer and Method

(71) Applicants: Cynthia Conner, Cambria, CA (US); Lisa Jo Wright, Cambria, CA (US)

(72) Inventors: Cynthia Conner, Cambria, CA (US); Lisa Jo Wright, Cambria, CA (US)

(21) Appl. No.: 17/134,648

(22) Filed: Dec. 28, 2020

Related U.S. Application Data
(60) Provisional application No. 63/078,936, filed on Sep. 16, 2020.

(51) Int. Cl. A61L 2/20; A61L 2/26; A61L 2/07; A61L 2/10

(52) U.S. Cl. CPC A61L 2/208; A61L 2/26; A61L 2/07; A61L 2202/17; A61L 2202/122; A61L 2202/121; A61L 2202/11; A61L 2/10

(57) ABSTRACT

This present invention relates to a system and method for cleaning and sanitizing cards, passes, badges and the like which have been used by individuals in one or more transactions and which may have been exposed to viruses, germs, bacteria, microbes and the like. The system includes an enclosure for treating the cards at an elevated temperature and/or with UV light in order to clean and sanitize the cards. The system allows for cards to be quickly placed back in circulation or use after the sanitizing treatment.

### CROSS-REFERENCE TO RELATED APPLICATION

The present application claims priority to, and the benefit of, U.S. Provisional Application No. 63/078,936, which was filed on Sep. 16, 2020 and is incorporated herein by reference in its entirety.

### FIELD OF THE INVENTION

The present invention relates to the field of cleaning and sanitizing equipment. More specifically, the device and method of the present invention relate to the cleaning and sanitizing of credit cards, key cards, membership or loyalty cards, ID cards, and the like that may presented as part of a transaction, or in order to gain entry into a facility or unlock a door, locker or other secured area. Accordingly, the present specification makes specific reference to the presently described invention, it is to be appreciated that aspects of the present invention are also useable with other like applications, devices and methods of manufacture.

### BACKGROUND OF THE INVENTION

Today, there are numerous cards, fobs, passes, badges, vouchers and other collateral devices which are exchanged between or carried by individuals in order to provide information in connection with certain activities, such as presenting payment, unlocking a door, presenting an identification card and the like. Because of the frequent exchange of such cards and devices amongst many different individuals, the cards and devices can pass germs, viruses, bacteria, pathogens and the like to other individuals when cards are exchanged, or even when they are simply used, such as inserting the card into a card slot or swiping a card through a card reader to gain access into a room.

In fact, research has shown that bacteria, germs, viruses and the like can remain on plastic, metal or glass surfaces for three to five days, and maybe as long as up to seven or nine days. As such, when an individual uses or carries a card, having to put a card aside, essentially taking it out of commission for a week or more, particularly when it is something like a credit card or identification card is simply unacceptable, as the person will need to use the card again prior to the time that any bacteria, germs, viruses and the like will have expired.

One such card, a key card, has become quite popular in recent years, and is now regularly used in hotels, offices, buildings and the like to gain access to a room or unlock a door to get into a secured area or building. Not only does the key card have the potential to become contaminated from being handled by various users, but it may also be exposed to bacteria, germs, viruses and the like when it is inserted into the key card slot which may, for example, be contaminated from coming into contact with other key cards. With respect to hotel key cards, after the guest checks out, the key card is usually placed in a container for later reuse, or is often immediately used by the hotel personnel for the next guest once it is reencoded with a different room number.

However, during times of disease outbreaks, such as during the cold and flu season or an epidemic or pandemic, the cards need to be cleaned and sanitized prior to the next use in order to avoid the spread of germs and bacteria from one individual to another. If there is no convenient way to efficiently and effectively clean the key cards, then the key cards may simply be discarded, thereby creating unnecessary waste, cost and increasing the carbon foot print of the business. Alternatively, the situation may require the hotel or motel to increase the total number of key cards that they have available for encoding due to the delay in the disposal, recycling or reuse of the key cards.

Having a convenient way to quickly clean, sanitize and/or sterilize hotel key cards is needed, as otherwise each container of cards collected must be labeled with the day collected and the cards would then be set aside for a week or more until the germs or bacteria have died, or are no longer present on the cards, and the cards are once again safe to use. In addition, guests can then be assured that the hotel is complying with relevant safety and cleanliness standards and that their stay should be relatively germ free.

Therefore, there exists a long felt need in the art for an improved and convenient to use card cleaning, sanitizing and/or sterilizing system which would provide commercial institutions, such as hotels, motels and other businesses, an efficient and effective way to process cards, badges, passes, identification cards and the like so as to stop the spread of germs, viruses and bacteria. There is also a long felt need in the art for a device for personal or residential use that could accomplish the same objectives as those found in commercial establishments, but on a smaller scale. For example, an individual could use the personal or residential version of the device to clean and sanitize his or her personal key or access cards that were used during the day without having to wait longer periods of time in order to be able to safely use the card again, and without the fear of bringing any germs, bacteria, viruses or the like that may be present on the cards into the home. Moreover, there is a long felt need in the art for a method of cleaning cards, badges, passes, identification cards and the like in an effective and efficient manner, with minimal effort on the part of the user, and without exposing the user to said contamination. Finally, there is a long felt need in the art for a card sanitizing device that is relatively inexpensive to manufacture, and that is safe and easy to use.

The present invention relates generally to an improved system for cleaning various cards, key fobs and other transaction related collateral which an individual may use in connection with processing payments, gaining access to a room or facility, showing identification and other instances where a card or fob may be present. More specifically, the system is comprised of a card sanitizing device and related method that employs a unique cleaning solution, relatively high temperature and/or the presence of ultraviolet (UV) light to kill off substantially all of the germs, bacteria, viruses and the like that may be present on the card or fob in a relatively short period of time. In this manner, the improved and convenient card sterilizing, cleaning and sanitizing device of the present invention accomplishes all of the forgoing objectives, and substantially departs from the concepts and designs of the conventional processes for ensuring safety of users of such cards regardless of the application.

### SUMMARY OF THE INVENTION

The following presents a simplified summary in order to provide a basic understanding of some aspects of the disclosed innovation. This summary is not an extensive overview, and it is not intended to identify key or critical elements or to delineate the scope thereof. Its sole purpose is to present some general concepts in a simplified form as a prelude to the more detailed description that is presented later.

The card sanitizing system and method of the present invention is used for the cleaning and sanitizing of one or more cards, which can be obtained from a variety of sources and may have been contaminated with viruses, germs, bacteria, pathogens or the like. The present invention allows the cards to be quickly reintroduced into circulation after undergoing a brief cleaning session. More specifically, during the cleaning session the cards are exposed to at least one of a cleaning solution that is provided at an elevated temperature and/or an UV light source in order to effectively treat the surfaces of the card and remove up to 99.9% of any contaminants that may be present on the cards themselves. Through use of such a system, care can be taken that the individual receiving the card or cards will not become inadvertently contaminated by any germs, viruses, pathogens or bacteria that were previously present on the card surfaces.

In one exemplary embodiment of the presently described invention, a card cleaning and sanitizing system is presented that is comprised of a housing having a base portion and a lid. The base portion has a plurality of side walls and a bottom to form an enclosure, and the lid fits over the base portion. A cover is provided over the top of the enclosure and under the lid, wherein the cover has a plurality of slots with each slot sized and configured to receive a card to be cleaned and/or sanitized. A sanitizer system is disposed within the enclosure, and below the cover and the lid.

In a further exemplary embodiment of the present invention, a hotel key card sanitizing system is described, and includes a plurality of hotel key cards and an enclosure for sanitizing the hotel key cards. The enclosure is comprised of a reservoir for holding a fluid, and a top surface covering the enclosure. The top surface has a plurality of slots and a lid that fits over the enclosure and surface for securing the lid to the enclosure. A heating element or device is further provided for heating a cleaning or sanitizing solution to generate steam both in the enclosure and in the reservoir to clean and sterilize the hotel key cards.

In another exemplary embodiment of the present invention, a hotel key card sanitizing system is described, and includes a plurality of hotel key cards and an enclosure for sanitizing the hotel key cards. The enclosure is comprised of a UV light source, and a top surface covering the enclosure. The top surface has a plurality of slots and a lid that fits over the enclosure and surface for securing the lid to the enclosure. Once placed in the slots, the hotel key cards are exposed to the UV light source and effectively sanitized.

In yet a further exemplary embodiment of the presently described invention, a method for sanitizing contaminated cards, such as hotel key cards, is presented. The sanitizing method includes the initial step of providing a sanitizing system comprised of an enclosure for containing a reservoir and a steam generator. The enclosure further has a surface with a plurality of slots disposed evenly on the surface. Next, a plurality of cards is obtained with the cards having been previously used and contaminated. Then each of the plurality of cards is placed in each of the slots, and a cleaning session is started wherein each of the cards is sanitized.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1A illustrates a perspective view of one potential embodiment of the card sanitizing system of the present invention in accordance with the disclosed architecture;

FIG. 1B illustrates a perspective view of an alternative embodiment of the card sanitizing system of the present invention in accordance with the disclosed architecture and having a USB connector and a power cord;

FIG. 2A illustrates a key card becoming contaminated by contacting a key slot of a door lock;

FIG. 2B illustrates a perspective view of a previously contaminated card being inserted into the enclosure of one potential embodiment of the card sanitizing system of the present invention in accordance with the disclosed architecture;

FIG. 3 illustrates a partial perspective view of one potential embodiment of the card sanitizing system of the present invention in accordance with the disclosed architecture and having a heater component;

FIG. 4 illustrates a partial perspective view of one potential embodiment of the card sanitizing system of the present invention in accordance with the disclosed architecture and having a steam cleaning feature;

FIG. 5 illustrates a partial perspective view of a sanitized key card being removed from one potential embodiment of the card sanitizing system of the present invention in accordance with the disclosed architecture; and

FIG. 6 illustrates a block diagram of one potential method of using the card sanitizing system of the present invention in accordance with the disclosed architecture.

### DETAILED DESCRIPTION OF THE INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention or do not limit the scope of the invention. Additionally, an illustrated embodiment need not have all the aspects or advantages shown. Thus, in other embodiments, any of the features described herein from different embodiments may be combined.

The present invention, in one exemplary embodiment, comprises a card sanitizing system having an enclosure that can be used to quickly and effectively clean and sanitize contaminated cards, such as key cards, fobs, credit cards and the like. The sanitizing system uses a steam generator to heat a cleaning solution to an elevated temperature sufficient to kill bacteria, germs, viruses, harmful microbes and the like. The cleaning solution may include water or other components such as hydrogen peroxide, vinegar, sodium bicarbonate or the like to further enhance the effectiveness of the cleaning session. For example, through the addition of oxidizing agents, the surface of the cards may be sanitized by the oxidation created by the sodium bicarbonate or hydrogen peroxide. The sanitizing system of the present invention may also be equipped with a UV light source to further assist in the cleaning and sanitizing of the previously contaminated cards. Alternatively, the UV light source may replace the steam cleaning component altogether as the sanitizing mechanism.

Cards that are potentially contaminated may be encountered from a number of different situations including transactional circumstances, such as presenting a credit card for payment; passing an identification card to an inspector for airport screening or other identification requirements; or using entry cards for gaining access to a building, office, hotel room or the like.

As used herein, “cards” refers to a number of different types of products such as transactional cards, key cards, badges, passes, key fobs, tickets, entry cards and the like. Most common cards, such as a credit card, have a size of approximately 3.37 inches by 2.125 inches and a thickness of between 10 to 30 millimeters to conform to the ISO/IEC 7810 ID-1 standard, with about 30 millimeters being about the most common thickness due to the adaptation of various card receiving slots, card readers, ATMs, point of sale terminals, door locks and the like.

Each time that a card is used, it can become contaminated with germs, viruses, bacteria or other pathogens. The contamination can occur by passing the card from one individual to another or, alternatively, when a card is inserted into a key slot and the slot itself is contaminated, such as by a contaminated card that was previously inserted into the slot. Once contaminated, the virus, bacteria, germs or other pathogens can remain on the surface of the card from anywhere between three to nine days, depending on where the card is stored and the environment in which the card is situated. As it is inconvenient to have to wait such a long period of time in order to use a card for another transaction, having a system that can easily and effectively clean and sanitize a card provides a general benefit to the public.

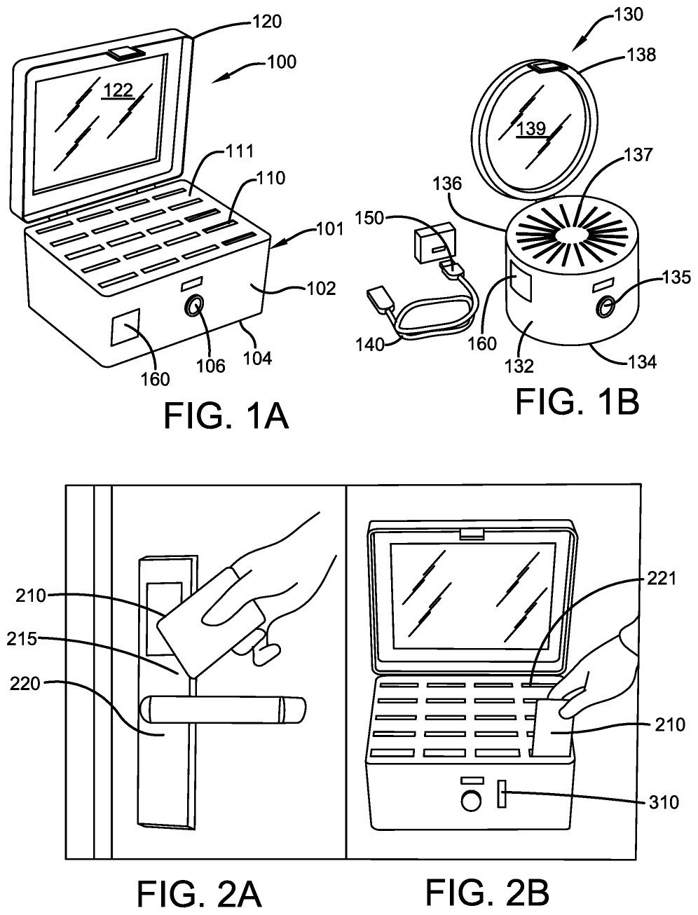

Referring now to the drawings, FIG. 1A illustrates a perspective view of one potential embodiment of the card sanitizing system 100 of the present invention in accordance with the disclosed architecture. More specifically, the card sanitizing system 100 includes a generally rectangular enclosure 101 having a plurality of walls 102, a bottom 104, a control 106, and a cover 110. The control 106 may be a simple on/off switch for powering the card sanitizing system 100, or may be more sophisticated and include different control settings, a timer and related alarm, and/or a sensor for sensing when the sanitization process is complete.

The cover 110 has a plurality of continuous openings or slots 111 therein, preferably presented in an orderly and spaced apart fashion, as shown in FIG. 1. Each of the plurality of slots 111 is sized and configured to receive a standard sized card as described above. The enclosure 101 is further comprised of a lid 120, which fits over the cover 110 and the enclosure 101 to seal the card sanitizing system 100 when the system is in operation and sanitizing the cards. The lid 120 may be repositionably attached to the enclosure 101 via a hinge (not shown), or it may be a separate component that may be simply placed on top of the enclosure 101 with an interference fit. In a preferred embodiment, the lid 120 may further comprise a transparent section or window 122, so that the user can view the interior of the enclosure 101 when in use to make sure that the same is operational.

FIG. 1B illustrates one potential alternative embodiment of the card sanitizing system 100 of the present invention and having a generally cylindrical enclosure 130, which may be used for smaller scale applications, such as for personal or residential use. Similar to enclosure 101, the cylindrical enclosure 130 is comprised of a sidewall 132, a bottom 134, a control 135, and a cover 136. The control 135 may be a simple on/off switch for powering the card sanitizing system 100, or may be more sophisticated and include different control settings, a timer, and/or a sensor for sensing when the sanitization process is complete.

The cover 136 has a plurality of continuous openings or slots 137 therein, preferably presented in an orderly and radially spaced apart fashion, as shown in FIG. 1. Each of the plurality of slots 137 is sized and configured to receive a standard sized card as described above. The enclosure 130 is further comprised of a lid 138, which fits over the cover 136 and the enclosure 130 to seal the card sanitizing system 100 when the system is in operation and sanitizing the cards. The lid 138 may be repositionably attached to the enclosure 130 via a hinge (not shown), or it may be a separate component that may be simply placed on top of the enclosure 130 with an interference fit. In a preferred embodiment, the lid 138 may further comprise a transparent section or window 139, so that the user can view the interior of the enclosure 130 when in use to make sure that the same is operational.

FIG. 1B further illustrates a power cord 140 so that the card sanitizing system 100 may be plugged in and powered by a conventional outlet, or it may include a USB 150 type of connection to allow for different charging and powering opportunities. Alternatively, the card sanitizing system 100 may further comprise a battery pack 160 to allow users to take the card sanitizing system 100 with them when traveling, or to use the system 100 remotely. If a battery is used, it may be a disposable battery or a rechargeable battery, and may be easily replaced or removed if necessary, for recharging. In one embodiment, the batteries can be selected from disposable batteries or rechargeable batteries; preferably double-A sized batteries in sufficient quantity to provide power to the sanitizing system 100. Suitable batteries include alkaline, nickel-cadmium, nickel-metal hydride, etc. Throughout this specification the terms “battery”, “battery pack”, and “batteries” may be used interchangeably to refer to one or more wet or dry cells or batteries of cells in which chemical energy is converted into electricity and used as a source of DC power. References to recharging or replacing batteries may refer to recharging or replacing individual cells, individual batteries of cells, or a package of multiple battery cells as is appropriate for any given battery technology that may be used.

FIG. 2B illustrates a perspective view of a previously contaminated card 210 being inserted into the slot 221 in the enclosure of one potential embodiment of the card sanitizing system 100 of the present invention in accordance with the disclosed architecture. More specifically, FIG. 2A displays a key card 210 becoming contaminated by insertion into a key slot 215 such as one may encounter at a door lock 220. The card 210 may become contaminated by the user handling the same, or by the insertion into the slot 215 where a previously contaminated card had been inserted. The card 210 is then inserted into a slot 221 of the card cleaning and sanitizing system 100 to undergo sanitizing treatment so that it can be reused.

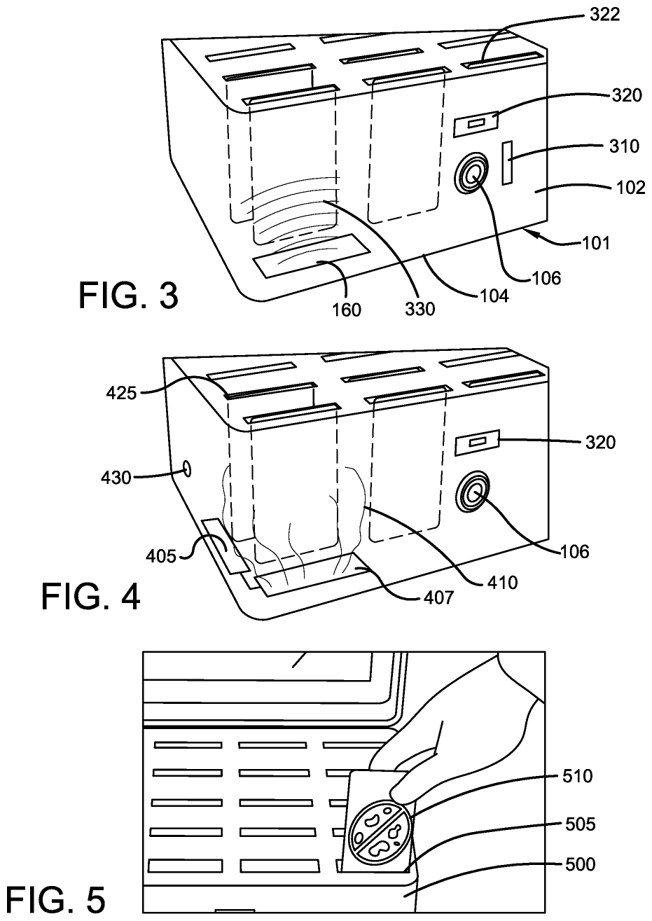

FIG. 3 illustrates a partial perspective view of one potential embodiment of the card sanitizing system 100 of the present invention in accordance with the disclosed architecture and having a heating element 330. More specifically, in this embodiment the sanitizing system 100 further comprises a lock 310 so that when a cleaning session is started, the enclosure 101 can be locked to secure the system until the sanitizing is completed. Sanitizing system 100 may further comprise an indicator 320, which may display visual indicia such as “open” or “closed” as well as colors, e.g. green or red, to show the status of the cleaning session, and whether it is safe to open the enclosure 110 and access the cards 210 that were subjected to the cleaning treatment. 322 again shows one of the many slots that may be present in the cover 110 or the enclosure into which the contaminated cards may be inserted, and the heating element 330 positioned on an interior of the enclosure 101 that is used to heat the cleaning solution stored in the enclosure 101 to create the steam that is used to sanitize the contaminated cards 210, as explained more fully bellow. The sanitizing system 100 of the present invention may also be equipped with a UV light source (not shown) to further assist in the cleaning and sanitizing of the previously contaminated cards, which may be powered by the battery pack 160, or electrical cord in communication with a wall outlet. Alternatively, the UV light source may replace the heating/steam cleaning component altogether as the sanitizing mechanism.

FIG. 4 illustrates a partial perspective view of one potential embodiment of the card sanitizing system 100 of the present invention in accordance with the disclosed architecture and having a steam cleaning feature. More specifically, steam 410 is generated by a steam generator 405, which includes one or more heating elements 330. The steam generator 405 is connected to a reservoir 407 which contains a cleaning solution, which is to be turned into gas when the temperature of the cleaning solution is heated to between 175° and 212° F. The steam 410 then fills the enclosure 101, and surrounds the contaminated cards 425 after the same have been inserted into the plurality of slots 420 in the cover. As previously stated, the cleaning solution may include water or other components such as hydrogen peroxide, vinegar, sodium bicarbonate or the like to further enhance its effectiveness. For example, through the addition of oxidizing agents, the surface of the cards may be sanitized by the oxidation created by the sodium bicarbonate or hydrogen peroxide. FIG. 4 also shows the electrical outlet port 430 which may be connected directly to a typical electrical outlet plug, or alternatively via a USB cable to a USB port.

FIG. 5 illustrates a partial perspective view of a sanitized key card 510 being removed from one potential embodiment of the card sanitizing system 100 of the present invention in accordance with the disclosed architecture. More specifically, FIG. 5 shows a fully cleaned and sanitized card 510 being withdrawn from the slot 505 of the enclosure 500. The cleaning session should last for at least 3 minutes after the steam has been generated, and the surrounding area in the enclosure 500 is filled with the heated steam and thereby maintaining a heated environment between 175° and 212° F. The steam may be provided by just heating water, deionized water or distilled water, or other components may be added to the water, including hydrogen peroxide in a percentage of solution ranging from 3 to 30 percent by volume. In addition, other components such as white or distilled vinegar may be included, as well as sodium bicarbonate, or baking soda as it is commonly known.

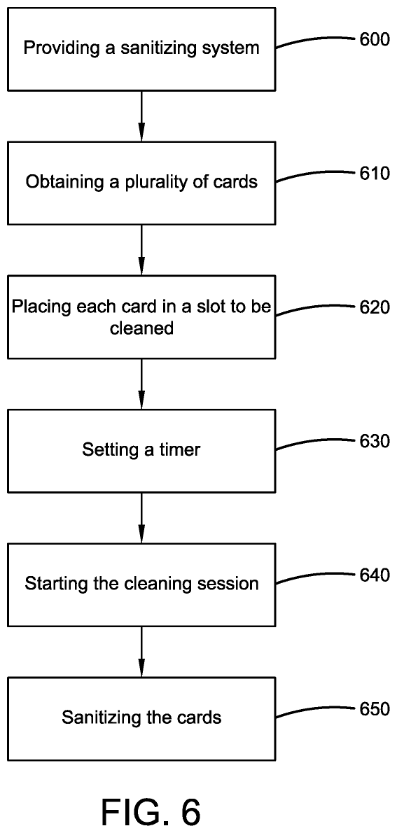

FIG. 6 illustrates a block diagram of one potential method of using the card sanitizing system of the present invention in accordance with the disclosed architecture. More specifically, at step 600 the sanitizing system described above is provided, and a plurality of contaminated cards to be cleaned is obtained at step 610. The cards may have been obtained, for example, at the front desk of a hotel when a guest returns his or her card after checking out of the hotel. At step 620, each contaminated card is placed into a slot in the cover of the enclosure. There are a number slots provided, and the number of slots can vary from a few slots to several dozen, depending on whether the device is being used by an individual or by a large institution that may receive many contaminated cards each day. At step 630, a timer is set for the cleaning session. The timer should run for a sufficient amount of time, not less than 3 minutes, to effectively clean and sanitize the cards that have been placed in the slots in the enclosure. At step 640, the cleaning session is started and can include subjecting the cards to steam and a cleaning solution, that may include hydrogen peroxide. In addition or alternatively, the cleaning session can include exposing the contaminated cards to a UV light source. Finally, at step 650, the cards are sanitized and cleaned and can be removed from the slots in the enclosure and recirculated for use.

In a further embodiment of the present invention, an activated oxygen or ozone system could the used to perform the cleaning and sanitizing within the enclosure. More specifically, a generator could be used to break down the chemical bond of common oxygen (O2) and allows it to recombine into a new molecule that has three atoms of oxygen instead of two. After the activated oxygen has been generated it will automatically decompose back to oxygen, typically within two hours.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “cards” are interchangeable and refer to cards, fobs, passes, tickets, badges and other transactional devices used by individuals that may be exposed to contamination.

Notwithstanding the forgoing, the components of improved and of the present invention can be of any suitable size and configuration as is known in the art without affecting the overall concept of the invention, provided that it accomplishes the above stated objectives. One of ordinary skill in the art will appreciate that the size, configuration and material as shown in the FIGS. are for illustrative purposes only, and that many other sizes and configurations are well within the scope of the present disclosure in order to meet the specific opportunity for which one is using the current invention.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A card cleaning and sanitizing system comprising:
a housing comprised of a base portion and a lid, wherein the base portion is comprised of a plurality of side walls and a bottom to form an enclosure and the lid fits over the enclosure;
a cover placed over a top of the enclosure and under the lid, wherein the cover is comprised of a plurality of slots with each of the plurality of slots is sized and configured to receive a card; and
a sanitizing device disposed within the enclosure and below the cover and the lid.

2. The card cleaning and sanitizing system of claim 1, wherein the sanitizing device comprises a steam generator.

3. The card cleaning and sanitizing system of claim 1, wherein the enclosure comprises a reservoir for holding a cleaning solution.

4. The card cleaning and sanitizing system of claim 3, wherein the cleaning solution is comprised of hydrogen peroxide in an amount ranging from 3 to 30 percent by volume.

5. The card cleaning and sanitizing system of claim 2, wherein the steam generator heats a cleaning solution to between 175° to 212° F. to create a steam.

6. The card cleaning and sanitizing system of claim 1, wherein the sanitizing device is comprised of a UV light source.

7. The card cleaning and sanitizing system of claim 1 further comprising a timer and an alarm to signal when a cleaning session is completed.

8. The card cleaning and sanitizing system of claim 1, wherein the plurality of slots may be configured to receive a credit card, a key card, a badge, a pass, an identification card, a key fob, a ticket and a combination thereof

9. The card cleaning and sanitizing system of claim 1, wherein the enclosure further comprises a lock to secure the card cleaning and sanitizing system during a cleaning session.

10. The card cleaning and sanitizing system of claim 1, wherein the enclosure further comprises an indicator that illustrates a status of a cleaning session.

11. A hotel key card sanitizing system comprising:
a plurality of hotel key cards;
an enclosure for sanitizing the plurality of hotel key cards, wherein the enclosure is comprised of a reservoir for holding a cleaning solution and a top surface covering the enclosure, wherein the top surface is comprised of a plurality of slots for receiving the plurality of key cards;
a lid that fits over the enclosure; and
a heating device for heating the cleaning solution and generating a steam disposed in the enclosure and connected to the reservoir to clean and sterilize the plurality of hotel key cards.

12. The hotel key card sanitizing system as recited in claim 11, wherein each of the plurality of hotel key cards has a size of approximately 3.37 by 2.125 inches, and a thickness of approximately 30 millimeters.

13. The hotel key card sanitizing system as recited in claim 11, wherein the cleaning solution is comprised of a hydrogen peroxide and a water, and further wherein the hydrogen peroxide ranges from 3 to 30 percent of the cleaning solution by volume.

14. The hotel key card sanitizing system as recited in claim 11 further comprising a UV light source for sanitizing the plurality of hotel key cards.

15. The hotel key card sanitizing system as recited in claim 11 further comprising an alarm and an indicator for alerting a user when a cleaning session is complete.

16. The hotel key card sanitizing system as recited in claim 15, wherein the alarm and the indicator are triggered after three minutes of exposure of the plurality of hotel key cards to the steam.

17. A method for treating contaminated cards, the method comprising the steps of:
providing a sanitizing system comprised of an enclosure for containing a reservoir and a steam generator, wherein the enclosure is comprised of a surface with a plurality of slots disposed evenly across the surface;
obtaining a plurality of cards, wherein the plurality of cards have been previously used and contaminated;
placing each of the plurality of cards in each of the plurality of slots;
starting a cleaning session; and
sanitizing each of the plurality of cards with a steam .

18. The method for treating contaminated cards as recited in claim 17, wherein the step of sanitizing includes exposing each of the plurality of cards to a cleaning solution at a temperature of between 175° to 212° F.

19. The method for treating contaminated cards as recited in claim 18, wherein the cleaning solution includes an amount of water and an amount of hydrogen peroxide.

20. The method for treating contaminated cards as recited in claim 18, including a further step of setting a timer after the step of placing each of the plurality of cards in each of the plurality of slots.
